(19) United States
(12) Patent Application Publication
(10) Pub. No.: US 2025/0144073 A1
(43) Pub. Date: May 8, 2025

(54) ANTI-CANCER, ANTI-INFLAMMATORY AND IMMUNOMODULATORY ACTIVITY OF R-CARVEDILOL AND METHODS OF USE

(71) Applicants: Ying Huang, Arcadia, CA (US); Bradley Tram Andresen, Upland, CA (US); Jinghua Jeffrey Wang, Aradia, CA (US); Vijay Kumar Nekkanti, Manchester, MO (US); Aysz Shahid, Pomona, CA (US)

(72) Inventors: Ying Huang, Arcadia, CA (US); Bradley Tram Andresen, Upland, CA (US); Jinghua Jeffrey Wang, Aradia, CA (US); Vijay Kumar Nekkanti, Manchester, MO (US); Aysz Shahid, Pomona, CA (US)

(21) Appl. No.: 18/739,283

(22) Filed: Jun. 10, 2024

Related U.S. Application Data
(63) Continuation of application No. 17/676,684, filed on Feb. 21, 2022, now abandoned.
(60) Provisional application No. 63/151,711, filed on Feb. 20, 2021.

(51) Int. Cl. A61K 31/403; A61P 9/04; A61P 9/10; A61P 9/12; A61P 35/00

(52) U.S. Cl. CPC A61K 31/403; A61P 9/04; A61P 9/10; A61P 9/12; A61P 35/00

(57) ABSTRACT

A novel prophylactic therapy for cancer, as well as therapy against DNA damage, inflammation and immunosuppression involves the β-blocker carvedilol, which is a racemic mixture consisting of two enantiomers, S- and R-carvedilol, in 1:1 ratio. S-carvedilol is a β-blocker, with a highly potent antagonizing activity against the β-adrenergic receptors, which is the main mechanism for the drug's pharmacological activity in treatment of high blood pressure and heart failure. Carvedilol—the racemic mixture—prevents ultraviolet radiation induced skin cancer by attenuating DNA damage, reducing inflammation and reversing immunosuppression. The non-β-blocking enantiomer R-carvedilol exhibits the same cancer preventive efficacy as the racemic carvedilol, without disturbing the cardiovascular system. Both carvedilol and R-carvedilol prevent chemical carcinogen-induced lung cancer development and lung inflammation.

### CROSS REFERENCE TO RELATED APPLICATIONS

This application claims the priority of Provisional Application No. 63/151,711 filed on Feb. 20, 2021, inventor Ying Huang et al., entitled “Prevention of skin carcinogenesis by the non-beta-blocking R-carvedilol enantiomer”. The entire disclosure of this provisional patent application is hereby incorporated by reference thereto in its entirety.

### STATEMENT REGARDING FEDERALLY SPONSORED RESEARCH OR DEVELOPMENT

Research reported in this invention was partly supported by the National Cancer Institute of the National Institutes of Health under Award Number R15CA227946 and Award Number R03CA256241 (PI: Ying Huang).

### FIELD OF THE INVENTION

The present invention relates to anti-cancer, anti-inflammatory and immunomodulatory activity of R-carvedilol and methods of use.

### BACKGROUND OF THE INVENTION

This invention was derived from several our previous studies indicating the cancer preventive effects of carvedilol, a third generation β-blocker approved in 1995 by the US FDA. Carvedilol is a receptor subtype non-selective β-blocker. Carvedilol's cancer preventive activity was indicated in a large population-based cohort study published in 2015, in which long-term carvedilol use significantly reduced cancer risk across all cancer types examined (Lin C S, 2015). In preclinical studies, carvedilol prevented chemical carcinogen-induced skin hyperplasia (Chang A, 2015) and UV-induced skin cancer development in mice (Huang K M, 2017). Mechanistic studies showed that carvedilol attenuated UV-induced DNA damage and activated oncogenic pathways including PI3K/AKT, MAPK/ERK, AP-1, COX-2 and NF-KB (Huang K M, 2017; Cleveland K H, 2018). There are ˜30 β-blockers of different receptor affinity and we examined 16 of them and found that only a small subset effectively prevented epidermal growth factor (EGF)-induced neoplastic transformation of mouse epidermal cells and carvedilol showed the highest potency (Cleveland K H, 2019).

Although carvedilol is a safe drug for long-term use in patients with cardiovascular conditions, its most pronounced effect is to slow the heart rate and thereby reduce cardiac output and blood pressure. The cardiovascular effects are a major obstacle for repurposing carvedilol as a cancer preventive agent since cancer prevention is an option offered for healthy individuals without cardiovascular disorders. To our surprise, our studies indicate that carvedilol prevents skin cancer independently of β-blockade (Cleveland K H, 2019). Importantly, carvedilol is a racemic mixture consisting of the β-blocking S-carvedilol and non-β-blocking R-carvedilol in 1:1 ratio. The non-β-blocking R-carvedilol demonstrated the same cancer-preventive activity as S-carvedilol in vitro and in vivo (Liang S, 2021). This work is featured on the cover of May, 2021 issue of Cancer Prev Res and a provisional US patent application has been filed by our University given its innovative nature and potential for clinical translation (63/151,711).

R-carvedilol has been used clinically in the racemic mixture for decades and R-carvedilol alone has been dosed in healthy human volunteers without major adverse effects (Stoschitzky K, 2001). Furthermore, oral R-carvedilol (1.6 mg/kg) did not alter heart rate or blood pressure in mice (Zhang J, 2015) and higher oral dose at 32 mg/kg did not alter blood pressure (FIG. 1). Therefore, using R-carvedilol for cancer prevention, the β-blockade-associated side effects can be avoided.

Since cancer prevention requires safe and tolerable interventions that can be given for long term, repurposing FDA-approved drug has great potential. Data disclosed in this invention indicate that the obstacle for repurposing carvedilol as a preventive agent, i.e., the cardiovascular effects, can be overcome by the use of R-carvedilol. Thus, it is a highly significant and novel idea to further develop R-carvedilol for cancer prevention. This invention is focused on prevention of non-melanoma skin cancer and lung cancer, but R-carvedilol can be similarly used to prevent other types of cancer.

### Object of the Invention

The invention involves methods for topical or oral administration of carvedilol or R-carvedilol to healthy individuals with increased risks of cancer, e.g., individuals with white skin tone, heavy smokers, and/or individuals with weakened immune system.

### SUMMARY OF THE INVENTION

The present invention provides a novel prophylactic therapy for cancer. The β-blocker carvedilol is a racemic mixture consisting of two enantiomers, S- and R-carvedilol, in 1:1 ratio. S-carvedilol is a β-blocker, with a very potent antagonizing activity against the β-adrenergic receptors. However, R-carvedilol is not a β-blocker. We discovered that carvedilol (the racemic mixture) prevents ultraviolet (UV) radiation induced skin cancer by attenuating DNA damage, reducing inflammation and reversing immunosuppression. Importantly, our recent studies indicate that the non-β-blocking enantiomer R-carvedilol exhibits the same cancer preventive efficacy as the racemic carvedilol, without disturbing the cardiovascular system. Furthermore, both carvedilol and R-carvedilol prevent chemical carcinogen-induced lung cancer development. Thus, this invention provides valuable information to support repurposing R-carvedilol as a novel prophylactic medication for cancer.

Other objects and advantages of the present invention will be more readily apparent from the following detailed description when read in conjunction with the accompanying drawings.

### BRIEF DESCRIPTION OF THE DRAWINGS

FIG. 1 is a graph depicting systolic blood pressure (SBP) of mice (males) treated with carvedilol, R-carvedilol, and a control in drinking water with respect to time measured in days.

FIG. 2 is a bar graph showing ear weight difference with respect to mouse treatment groups, the groups being labeled as Neg. Ctr., Post Ctr., UV, NS398+UV, CAR(L)+UV, and R-CAR(H)+UV.

FIG. 3 is a bar graph showing ear weight difference with respect to the groups of FIG. 2.

FIG. 4 is a graph showing Normalized FL3 intensity as intracellular uptake of T-CAR in B16-F10 cells with respect to time in minutes.

FIG. 5 is a graph showing cell viability (% control) demonstrating effects of T-CAR and free carvedilol on cell viability of B16-F10 cells, with respect to concentration.

FIG. 6 is a graph showing total tumor volume demonstrating the effects of T-CAR on B16-F10 bearing mice, with respect to mouse treatment groups including a control, free CAR L, free CAR H, Plain, and T-CAR.

FIG. 7 is a graph of total volume demonstrating combined treatment with T-CAR and αPD-1 showed better tumor inhibition (day 12), with respect to the mouse treatment groups control, T-CAR, RMP1-14, and T-CAR+RMP1-14.

FIG. 8 is a graph demonstrating combined how T-CAR and anti-PD-1 inhibit IL-1β (RT-PCR) with respect to group A: group 1, control; B: group 4, T-CAR+RMP1-14.

FIG. 9 is a graph demonstrating combined T-CAR and anti-PD-1 inhibit COX-2 mRNA expression (RT-PCR) with respect to A: group 1, control; B: group 4, T-CAR+RMP1-14.

FIG. 10 shows photographs of a control and a combined T-CAR and anti-PD-1 inhibit Ki-67 tumor marker (IHC): A: group 1, control; B: group 4, T-CAR+RMP1-14.

FIG. 11 is a bar graph demonstrating comparison on different groups of the effects of the racemic carvedilol, S- and R-carvedilol on B(a)P-induced early-stage tumor marker LDH of lung.

FIG. 12 is a bar graph demonstrating comparison on different groups of the effects of the racemic carvedilol, S- and R-carvedilol on B(a)P-induced early-stage tumor marker LPO of lung.

FIG. 13 is a set of images demonstrating effects of carvedilol on B(a)P induced histological changes of lung, and specifically demonstrating H&E staining of mouse lung sections to examine the morphology of the lung (x40 or x20 H&E staining) with the following groups: A: Control, B: B(a)P only, C: CUR+B(a)P, and D: CAR+B(a)P.

FIG. 14 is a set of images showing effects of carvedilol on B(a)P induced COX-2 expression. IHC analysis of Cox-2 (40x) for the groups: A: Control, B: B(a)P only, C: CUR+B(a)P, and D: CAR+B(a)P.

FIG. 15 is a bar graph demonstrating tumor multiplicity with regard to effects of CAR and R-CAR on long term B(a)P-induced lung carcinogenesis, for a various groups.

FIG. 16 is a graph representing tumor volume for different groups with respect to days after tumor innoculation, demonstrating effects of oral carvedilol and R-carvedilol on tumor growth of triple negative breast cancer 4T1 as monotherapy.

FIG. 17 is a graph representing tumor volume for different groups with respect to days after tumor innoculation, demonstrating effects of carvedilol alone or in combination with anti-PD1 therapy on the growth of 4T1 in mice.

FIG. 18 is a graph representing tumor volume for different groups with respect to days after tumor innoculation, demonstrating effects of R-carvedilol alone or in combination with anti-PD1 therapy on the growth of 4T1 in mice.

### DETAILED DESCRIPTION OF THE INVENTION

The present invention involves methods for topical or oral administration of carvedilol or R-carvedilol to healthy individuals with increased risks of cancer, e.g., individuals with white skin tone, heavy smokers, and/or individuals with weakened immune system.

The present invention provides a novel prophylactic therapy for cancer. The β-blocker carvedilol is a racemic mixture consisting of two enantiomers, S- and R-carvedilol, in 1:1 ratio. S-carvedilol is a β-blocker, with a very potent antagonizing activity against the β-adrenergic receptors. However, R-carvedilol is not a β-blocker. The inventors have discovered and demonstrated that carvedilol (the racemic mixture) prevents ultraviolet (UV) radiation induced skin cancer by attenuating DNA damage, reducing inflammation and reversing immunosuppression. Importantly, the recent studies by the inventors indicate that the non-β-blocking enantiomer R-carvedilol exhibits the same cancer preventive efficacy as the racemic carvedilol, without disturbing the cardiovascular system. Furthermore, both carvedilol and R-carvedilol prevent chemical carcinogen-induced lung cancer development. Thus, this invention provides valuable information to support repurposing R-carvedilol as a novel prophylactic medication for cancer.

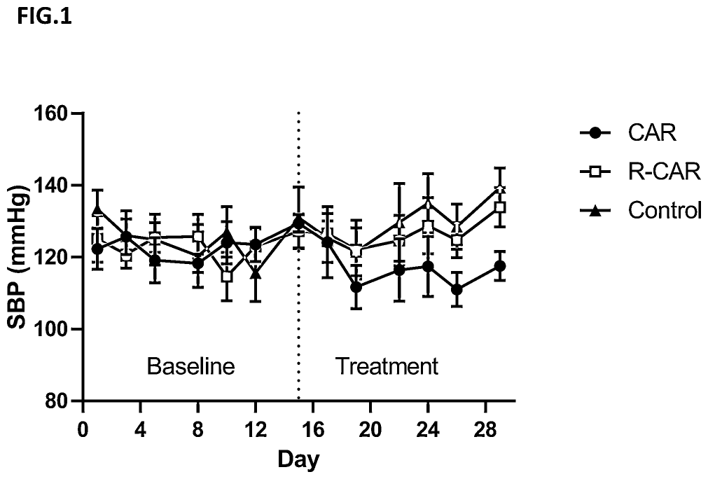

FIG. 1 shows systolic blood pressure (SBP) of mice (males) treated with carvedilol or R-carvedilol in drinking water (estimated dose 32 mg/kg) for 2 weeks. 178 μg/mL carvedilol and R-carvedilol in the drinking water of the mice resulted in a mean plasma concentration (and 95% confidence interval) of 32.29 ng/ml (16.71 to 47.74) and 25.13 ng/ml (7.81 to 42.46), respectively. Although the data did not show any correlation between plasma concentration and blood pressure response, only racemic carvedilol statistically reduced mean systolic blood pressure from control conditions (P=0.0367).

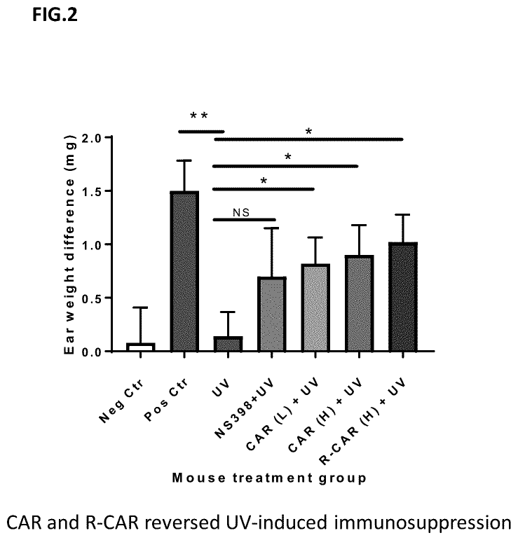

FIG. 2 shows carvedilol (CAR) and R-carvedilol (R-CAR) reversed UV-induced suppression of CHS. Mice were treated with NS398, CAR or R-CAR (L: 1 mg/kg; H: 10 mg/kg) i.p. 2 hr before UV exposure (224 mJ/cm²). 24 h after, mice were sensitized with DNFB on back. After 7 days, the right ears were challenged with DNFB. 24 h after, 5 mm punch biopsies were obtained from both ears and weighed. The data are the mean±SE difference in ear biopsy weights (right-left) from n=5 mice (male). *: P<0.05; **: P<0.01.

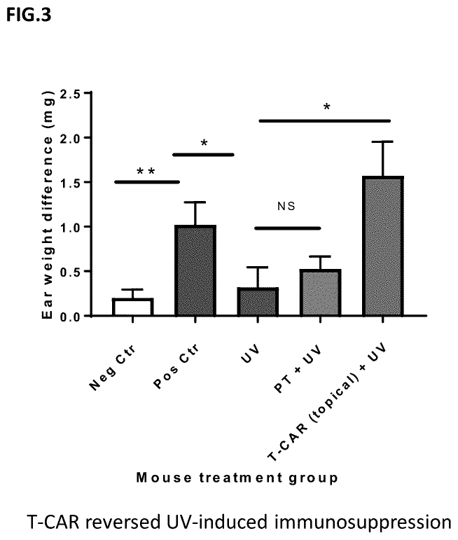

FIG. 3 shows effects of topical T-CAR on UV-induced immunosuppression. The data listed are the mean±SE difference in ear biopsy weights (Right-Left) from n=5 mice (male). *: P<0.05; **: P<0.01.

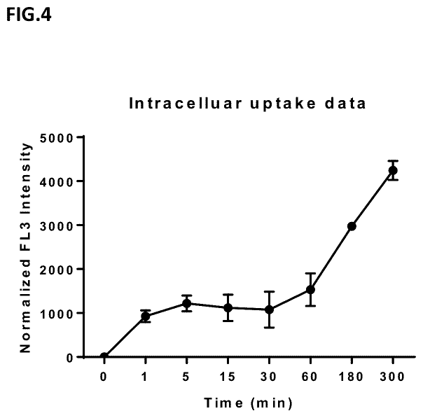

FIG. 4 shows intracellular uptake of T-CAR in B16-F10 cells. B16-F10 cells (1×10⁵) were plated in 12-well plates. After overnight incubation, the cells were incubated with DiI labeled T-CAR (4 ug/mL). After washing with PBS, the cells were trypsinized and the fluorescent intensity of cells at time points (1, 5, 15, 30, 60, 180 and 360 min) was determined by FACS analysis.

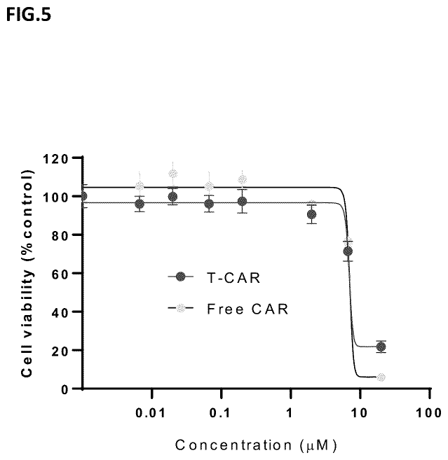

FIG. 5 shows effects of T-CAR and free carvedilol on cell viability of B16-F10 cells. The drugs were examined for their dose-dependent effects on the cytotoxicity (by SRB assay). Data were normalized to their respective controls.

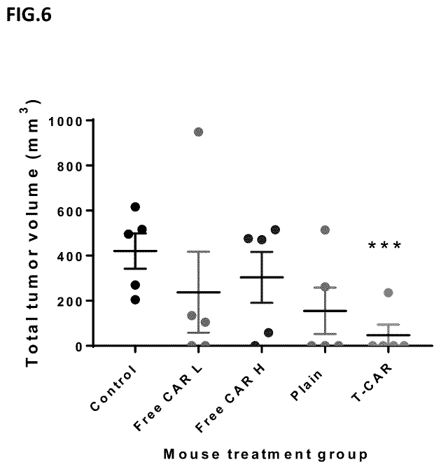

FIG. 6 shows effects of T-CAR on B16-F10 bearing mice. Comparison of the efficacy of free drug and T-CAR (day 14). One day after tumor inoculation (two tumors per mouse, mice received free drug or T-CAR daily.

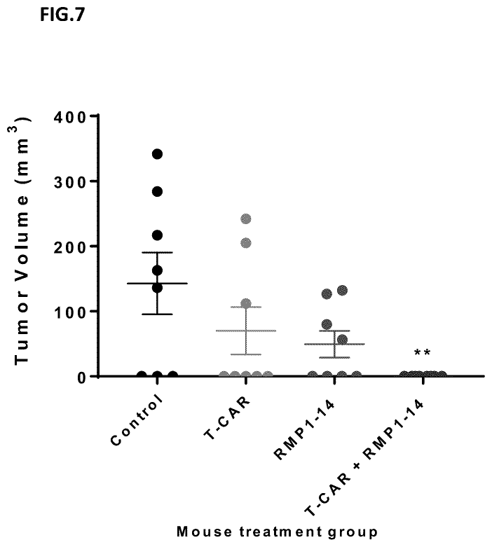

FIG. 7 shows combined treatment with T-CAR and αPD-1 showed better tumor inhibition (day 12). One day after tumor inoculation (one per mouse), mice received free drug or T-CAR daily, 200 μg RMP1-14 twice a week, or combination.

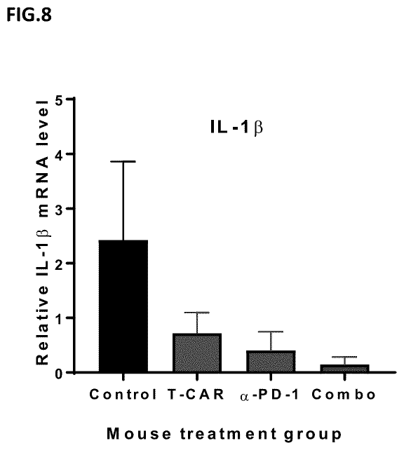

FIG. 8 shows combined T-CAR and anti-PD-1 inhibit IL-1β (RT-PCR). A: group 1, control; B: group 4, T-CAR+RMP1-14. Tumor collected at day 20 after inoculation.

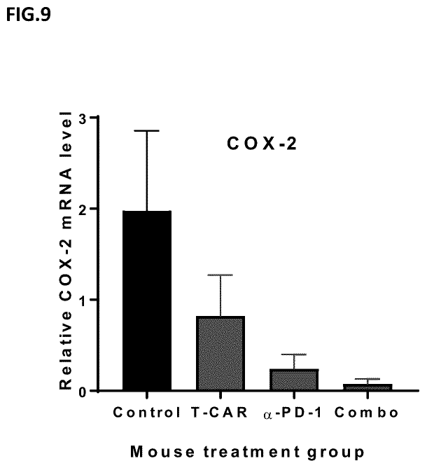

FIG. 9 shows combined T-CAR and anti-PD-1 inhibit COX-2 mRNA expression (RT-PCR). A: group 1, control; B: group 4, T-CAR+RMP1-14. Tumor collected at day 20 after inoculation.

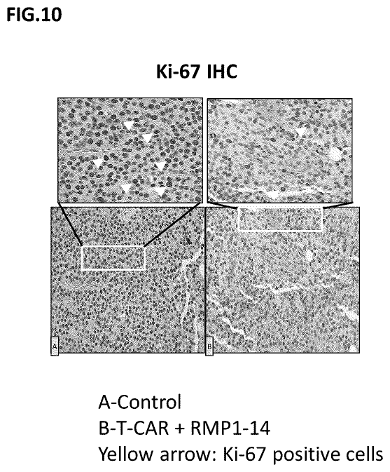

FIG. 10 shows combined T-CAR and anti-PD-1 inhibit Ki-67 tumor marker (IHC): A: group 1, control; B: group 4, T-CAR+RMP1-14. Tumor collected at day 20 after inoculation.

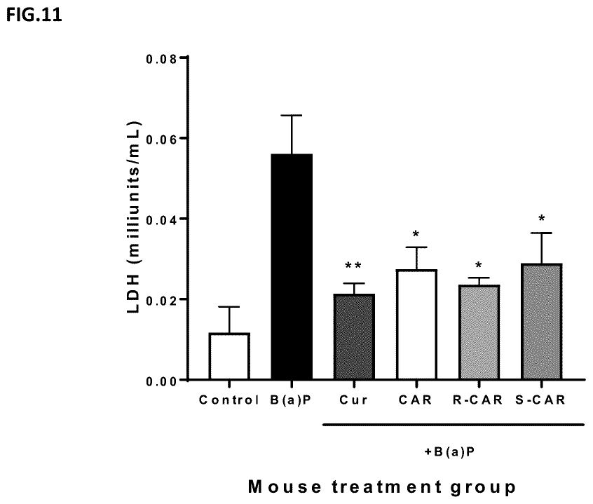

FIG. 11 shows a comparison of the effects of the racemic carvedilol, S- and R-carvedilol on B(a)P-induced early-stage tumor marker LDH of lung.

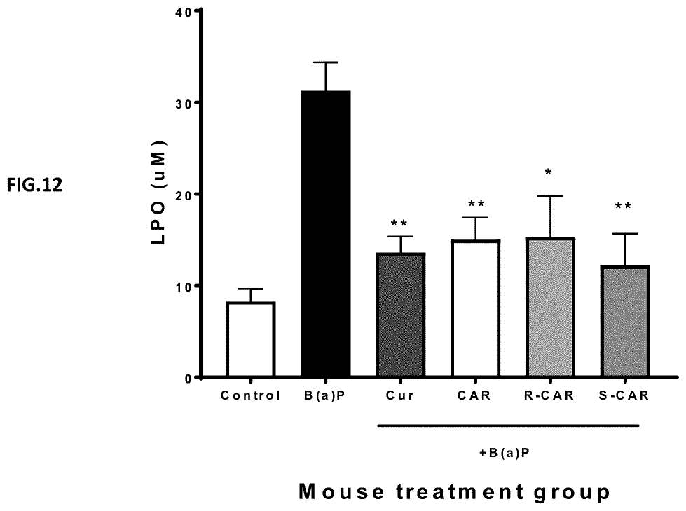

FIG. 12 shows a comparison of the effects of the racemic carvedilol, S- and R-carvedilol on B(a)P-induced early-stage tumor marker LPO of lung.

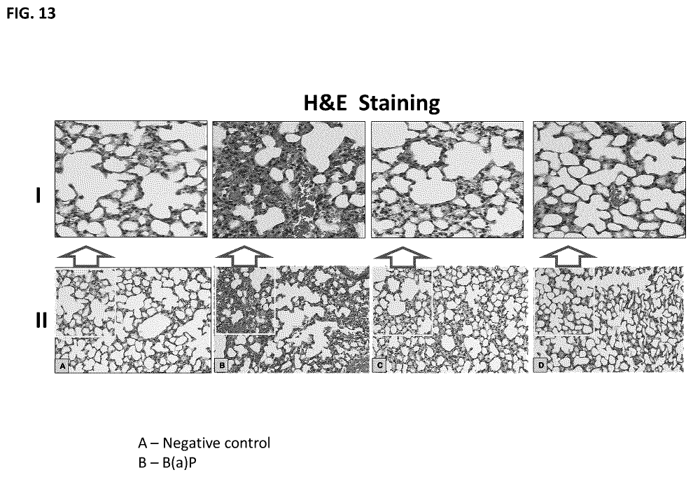

FIG. 13 shows effects of carvedilol on B(a)P induced histological changes of lung. H&E staining of mouse lung sections to examine the morphology of the lung (x40 or x20 H&E staining). A-Control, B-B(a)P only, C-CUR+B(a)P, D-CAR+B(a)P.

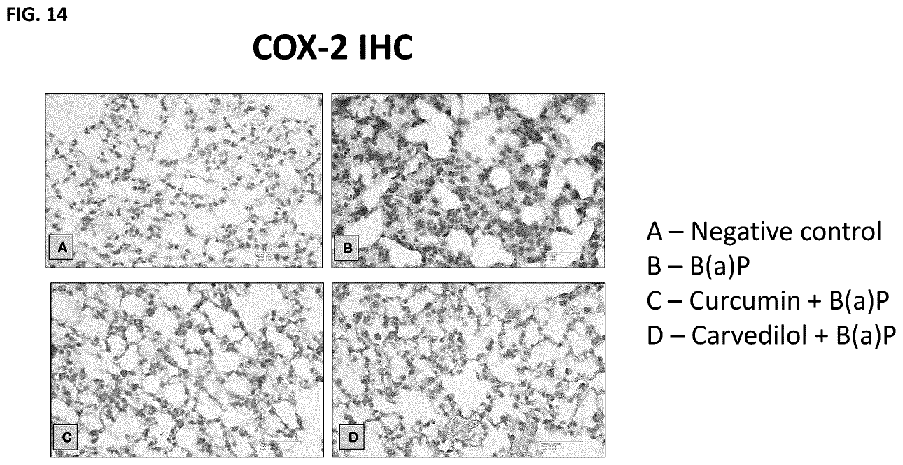

FIG. 14 shows effects of carvedilol on B(a)P induced COX-2 expression. IHC analysis of Cox-2 (40x). A-Control, B-B(a)P only, C-CUR+B(a)P, D-CAR+B(a)P.

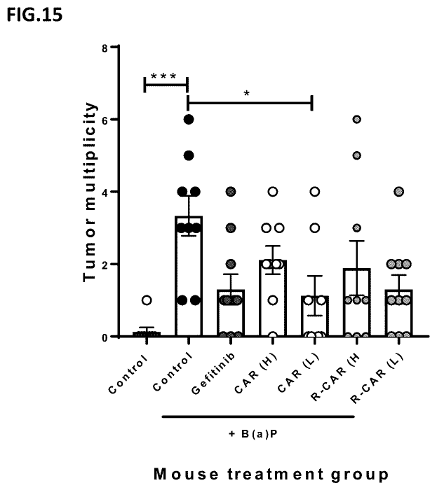

FIG. 15 shows effects of CAR and R-CAR on long term B(a)P-induced lung carcinogenesis. Data plotted are mean+SE; n=8-10. *: p<0.05; ***: p<0.001.

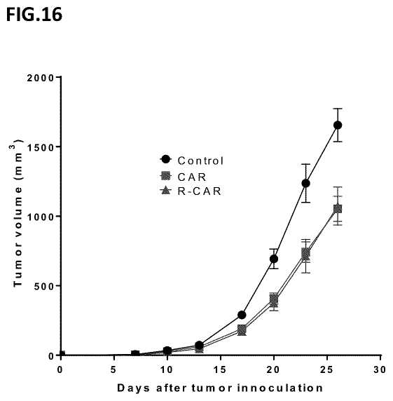

FIG. 16 shows effects of oral carvedilol and R-carvedilol on tumor growth of triple negative breast cancer 4T1 as monotherapy.

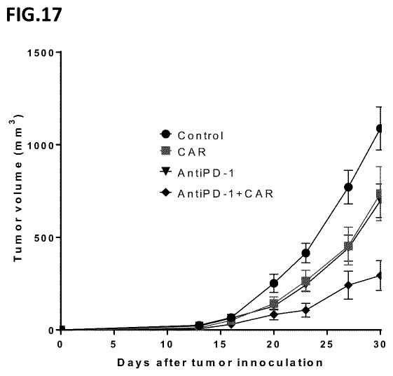

FIG. 17 shows effects of carvedilol alone or in combination with anti-PD1 therapy on the growth of 4T1 in mice.

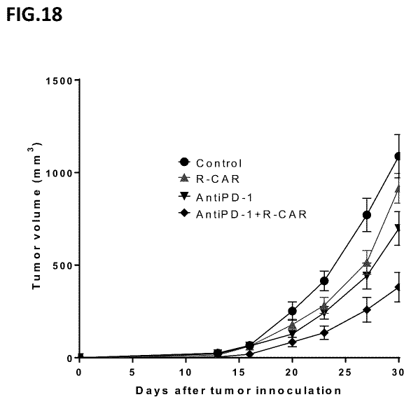

FIG. 18 shows effects of R-carvedilol alone or in combination with anti-PD1 therapy on the growth of 4T1 in mice.

### Topical Drug Delivery of R-Carvedilol

Due to a significant degree of first-pass metabolism, the bioavailability for orally administered carvedilol is only 24%. Therefore, a larger dose and high frequency are required to achieve the minimum effective concentration. Topical drug delivery has shown significant advantages in clinical practice for drugs targeting to the skin, which not only avoids first-pass metabolism but also reduces systemic side effects. The present inventors' data indicate that topically applied carvedilol loaded nano-transfersome (T-CAR) produced a controlled and prolonged skin drug delivery and enhanced drug efficacy. Importantly, a novel T-CAR topical gel demonstrated efficacy in preventing UV-induced skin cancer development in mice, but with negligible systemic absorption. Studies conducted based on topical delivery of carvedilol based on transfersomes proved the concept that topical application of carvedilol as well as R-carvedilol is able to effectively prevent skin cancer while avoiding side effects that are possibly associated with their cardiovascular disturbance.

Thus, topical route has been investigated as a major approach in the application of R-carvedilol for clinical skin cancer prevention. In order to prepare for clinical studies, we prepared three types of transfersome-based formulation for R-carvedilol using different amount of phospholipid (soy phosphatidylcholine, SPC) or edge activator (tween-80). The particle sizes, zeta potential, encapsulation efficiency and stability for these transfersomes were very similar to those of T-CAR. The in vitro penetration and skin retention studies using Franz diffusion cells and porcine ear skin showed that the formulation in the ratio of 1:3:0.5 (drug: SPC: tween-80) gave the highest skin retention, while the free drug R-carvedilol dissolved in water or Polyethylene Glycol (PEG) 400 showed very little skin retention and permeation. This confirms that nano-transfersomes provide a promising delivery system for both carvedilol and R-carvedilol. Thus, this selected transfersomal formulation for R-carvedilol, named “T-RCAR”, will be further characterized in preclinical model systems and examined in clinical trials in the future.

Instead of nanoparticle-based topical delivery, the present inventors have explored other methods of skin delivery of drugs. They investigated the effects of natural penetration enhancers on the ex vivo permeation of carvedilol across rat skin. The phosphate buffered saline (pH 7.4) containing 40% v/v polyethylene glycol 400 was used as a control, as in this formulation the solubility of drug is greatly increased in comparison with the saline only, whereas the skin permeation of R-carvedilol is minimal. The drug formulations containing carvedilol or R-carvedilol (200 μg/mL) (1 mL) was loaded on Franz diffusion cells using excised rat dorsal skin (with hair removed). The permeation of carvedilol in the control formulation showed low permeation (0.4+/−0.07 μg). However, the permeation of carvedilol in a formulation containing 5% w/v concentration of D-limonene showed significantly increased permeation (1.6+/−0.25 μg). D-limonene is isolated from orange peel, and is a cheap and safe option to delivery carvedilol (same for R-carvedilol) for skin cancer prevention. D-limonene is present in many skin commercial products such as perfumes and essential oils. Therefore, this invention provides another novel approach of making skin targeting formulation for both carvedilol and R-carvedilol. This approach of making skin formulation is not only safe but also easier to scale up to industry level for making large amount.

### Oral Drug Delivery of R-Carvedilol

An alternative route of delivery of R-carvedilol is oral delivery, which is the original route. The long acting carvedilol is commercially available (COREG CR), which is an extended-release capsule intended for once-daily administration. The COREG CR capsules have 10, 20, 40 or 80 mg once daily. Coreg CR is a prescription medicine used to treat the symptoms of Congestive Heart Failure, Chest Pain (Angina pectoris), Left Ventricular Dysfunction following Myocardial Infarction and High Blood Pressure (Hypertension). Coreg CR may be used alone or with other medications. However, there is no commercial R-carvedilol product. This invention contemplates use of oral R-carvedilol capsules for twice daily (immediate release) or once daily (slow release) use for the repurposed use in cancer chemoprevention. Since R-carvedilol does not modulate the cardiovascular parameters such as blood pressure, the R-carvedilol oral capsules are anticipated to provide a safe and effective way to prevent many types of cancers.

### EXAMPLE 1. R-CARVEDILOL PREVENTS SKIN CANCER

R- and S-carvedilol produced nearly identical inhibitory effects on EGF-induced JB6 transformation. The IC₅₀ values for R- and S-carvedilol in this assay were similar to that of the racemic carvedilol. In an acute UV induced skin damage and inflammation model induced with a single dose of UV (300 mJ/cm²), topical treatment with R-carvedilol (0.1, 1 or 10 μM in acetone as a vehicle), applied immediately after the UV, attenuated mouse skin edema and reduced epidermal thickening, Ki-67 staining, COX-2 protein, IL-6 and IL-1β mRNA levels. In chronic UV induced skin carcinogenesis model, topical R-carvedilol (10 μM in acetone) applied 30 min before UV strongly inhibited skin tumor development, showing higher efficacy than carvedilol, e.g., carvedilol- and R-carvedilol treated mice showed 3- and 5-week delay in tumor formation compared to UV only controls, respectively. These data have been published (Liang S, 2021) and a provisional US patent application has been filed (63/151,711).

### EXAMPLE 2. CARVEDILOL AND R-CARVEDILOL OVERCOME IMMUNOSUPPRESSION

### 2.1. Systemic Administration of Carvedilol Prevented UV-Induced Immunosuppression in Mice

Immunosuppression induced by UV radiation is commonly investigated using the contact hypersensitivity (CHS) response to contact allergens such as the hapten dinitrofluorobenzene (DNFB). In SKH-1 hairless mice, a single dose UV at 224 mJ/cm² strongly suppressed the CHS to DNFB (P<0.01) (FIG. 2). A single dose i.p. injection of carvedilol (1 and 10 mg/kg) 2 hours before UV exposure reversed UV-induced CHS suppression, which was more effective than NS-398 (positive control, a COX-2 inhibitor, 1 mg/kg, i.p.) (FIG. 2). Thus, carvedilol's skin cancer preventive activity may be partly attributed to its inhibitory effects on UV-induced immunosuppression.

### 2.2. Systemic Administration of R-Carvedilol Prevents UV-Induced Immunosuppression

The mice were pretreated with single dose of R-carvedilol (10 mg/kg, i.p.) two hours before UV radiation, and were subject to UV exposure. R-carvedilol significantly reversed UV-induced CHS suppression, to the same degree as the racemic carvedilol (FIG. 2). Thus, R-carvedilol, although not a β-blocker, similarly reverses UV-induced immunosuppression. These data further confirm that carvedilol's skin cancer preventive activity may not be attributed to β-blockade and R-carvedilol can be used instead of carvedilol in cancer prevention.

### 2.3. Topical Administration of Carvedilol Prevented UV-Induced Immunosuppression in Mice

CHS assay was used to evaluate the effects of a topical delivery system of carvedilol developed by us, named “T-CAR”, on UV-induced immunosuppression. The mice were pretreated daily with the topical T-CAR gel containing 10 μM carvedilol (volume 200 μL), two days in a row, and subject to UV exposure (224 mJ/cm²), followed by a third dose of T-CAR immediately after the UV exposure. The T-CAR gel significantly reversed UV-induced CHS suppression, while the effects of the plain transfersome (PT, an empty transfersome gel without drug) was non-significant (FIG. 3). These data confirm that carvedilol's skin cancer preventive activity is partly attributed to its effects against UV-induced immunosuppression.

### EXAMPLE 3. CARVEDILOL AND R-CARVEDILOL SENSITIZED ESTABLISHED TUMORS IMMUNOTHERAPIES

Recently, immune checkpoint inhibitors have become the first line treatment for many types of cancer, including triple negative breast cancer and malignant melanoma. Pembrolizumab (KEYTRUDA) is a humanized antibody that blocks the interaction between PD-1 and its ligands such as PD-L1. Unfortunately, a substantial number of patients fail to respond to anti-PD-1 therapies. This unmet medical need demands novel drug combinations that are effective and safe. We have discovered that both carvedilol and R-carvedilol are able to enhance the efficacy of the anti-PD-1 based immunotherapies.

### 3.1. Topical Carvedilol Sensitized Melanoma to Anti-PD-1 Therapies

The carvedilol loaded transfersome, T-CAR, was evaluated in vitro and in vivo on melanoma growth. The transfersome labeled with the fluorescent dye Dil demonstrated time-dependent intracellular uptake into murine melanoma B16-F10 cells in culture (FIG. 4). T-CAR demonstrated similar in vitro cell growth inhibitory activity as the free drug on B16-F10 (FIG. 5). To examine the in vivo activity of T-CAR, in C57BL/6 mice bearing the B16-F10 tumor, the T-CAR gel as a topical monotherapy was examined. The efficacy of T-CAR was compared with free drug dissolved in acetone in male mice bearing B16-F10 (two tumors inoculated in each mouse). At dose of 10 μM, at day 14 of tumor inoculation, both T-CAR and free drug in acetone showed trend of tumor inhibition while free drug at 100 μM (CAR H) was less effective (FIG. 6). Only T-CAR at 10 μM showed significant tumor inhibition. Second study examined efficacy of topical T-CAR combined with anti-PD-1 (RMP1-14, 200 μg per mouse, i.p. twice per week) (one tumor was inoculated in each mouse). Although T-CAR alone (10 μM) inhibited tumor, combined with anti-PD-1, the effect was greater (FIG. 7): at day 12 after tumor inoculation, 63% of control, 38% of T-CAR, 50% in αPD-1 group carried tumors, while no visible tumor was seen in the combo group (P<0.05). Tumor mRNA expression of IL-1β and COX-2 was inhibited to a greater degree by combined therapy, as well as the proliferation marker Ki-67 (FIG. 8, FIG. 9, and FIG. 10).

### 3.2. Carvedilol and R-Carvedilol Sensitized Triple Negative Breast Cancer to Anti-PD-1 Therapies

To investigate the effect of carvedilol and R-carvedilol on breast cancer growth, the BalB/C mice were treated with the drugs (20 mg/kg/day) in drinking water for two weeks before the mice were inoculated with murine triple negative breast tumor 4T1 in the fourth left mammary fat pad. In mice treated with carvedilol and R-carvedilol, the tumor growth was significantly reduced (FIG. 16). Carvedilol and R-carvedilol showed the same degree of tumor inhibition in this model. The same experimental model was treated with carvedilol or R-carvedilol, in combination with the anti-PD-1 therapy. Both carvedilol and R-carvedilol enhanced the efficacy of anti-PD-1 monotherapy (FIG. 17 and FIG. 18). These results indicate that R-carvedilol may be also used for increasing the efficacy of anti-tumor immunotherapies.

### EXAMPLE 4. CARVEDILOL AND R-CARVEDILOL PREVENT LUNG CANCER DEVELOPMENT

### 4.1 Effects of Carvedilol, S- and R-Carvedilol on Short Term Benzo(a)Pyrene-Induced Lung Toxicity in Mice

Chronic exposure to carcinogens that are present in tobacco smoking or air pollution is a well-known cause of lung cancer. One of the most important strategies to manage lung cancer is chemoprevention, which uses synthetic or natural products to block or reverse lung cancer initiation, promotion, and progression. Benzo(a)pyrene (B(a)P), a polycyclic aromatic hydrocarbon, is a pro-carcinogen ubiquitously present as pollutants in the environment. It is mostly produced during cigarette smoking and is present in automobile exhausts, and causes lung toxicity and lung cancer after metabolic activation. A single dose B(a)P-induced lung toxicity study in mice was used to evaluate the protective effects of carvedilol. In male CD1/IGS mice, carvedilol was orally administered (20 mg/kg body weight) once a day for 7 days. The mice were then treated with a single dose B(a)P via oral gavage (125 mg/kg body weight) on day 7, 30 min after the drug treatment. Mice were sacrificed 24 hours after B(a)P treatment. As expected, B(a)P increased the plasma levels of lactate dehydrogenase (LDH) and Malondialdehyde (MDA) for lipid peroxides (LPO). Treatment with carvedilol, R- and S-carvedilol significantly attenuated LDH and MDA levels to a similar degree as curcumin (100 mg/kg, positive control) (FIG. 11 and FIG. 12). Histological analysis of lung section indicates that B(a)P caused dramatic inflammatory cell infiltration, interstitial and alveolar edema, vascular congestion and alveolar collapse, while lungs from mice treated with carvedilol exhibits a reduction in these morphological changes. Immunohistochemical (IHC) analysis indicates that B(a)P strongly induced the expression of COX-2, while treatment with carvedilol attenuated it. S- and R-carvedilol demonstrated the same degree of protection as the racemic carvedilol (FIG. 13 and FIG. 14).

### 1 4.1

### 4.2 Effects of Carvedilol and R-Carvedilol on Long Term B(a)P-Induced Lung Carcinogenesis in Mice

To examine the chemopreventive efficacy of carvedilol, we used an established lung carcinogenesis model in A/J mice induced by B(a)P. Two drug doses were examined: 20 mg/kg (H) and 3.2 mg/kg (L). The mice were randomly divided by body weight into seven groups at the beginning of the experiments. To induce lung tumor, mice were given a single intraperitoneal (i.p.) injection of B(a)P at 100 mg/kg body weight in 0.2 ml tricaprylin. The following are treatment groups: (1) vehicle control, n=8; (2) B(a)P only, n=9; (3) B(a)P+gefitinib 400 mg/kg, weekly oral gavage, positive control, n=10; (4) B(a)P+carvedilol 20 mg/kg drinking water, n=9; (5) B(a)P+carvedilol 3.2 mg/kg drinking water, n=8; (6) B(a)P+R-carvedilol 20 mg/kg drinking water, n=9; (7) B(a)P+R-carvedilol 3.2 mg/kg drinking water, n=10. The mice began the drug treatments 3 weeks before B(a)P treatment and terminated at 23 weeks after B(a)P treatment. During the course of treatment, there was no body weight loss for all the dosing groups, although B(a)P slightly reduced the weight but recovered in a week. Tumor multiplicity was determined by counting surface tumors in fixed lung under a dissecting microscope. B(a)P significantly induced lung tumor formation (100% mice developed tumor). Carvedilol at the lower dose significantly attenuated the tumor multiplicity (FIG. 15). The tumor incidence for gefitinib group was 70%. The tumor incidence was 89% and 50% for high and low dose carvedilol, respectively. The tumor incidence was 67% and 70% for high and low dose R-carvedilol, respectively. R-carvedilol showed the same trend of tumor inhibition as carvedilol. The lower dose appears to be more effective.

### EXAMPLE 5. CARVEDILOL, BUT NOT R-CARVEDILOL, AFFECTS BLOOD PRESSURE OF MICE

SKH-1 mice were given racemic carvedilol or R-carvedilol in drinking water for two weeks (32 mg/kg/day). Blood pressure was examined in mice utilizing a Kent CODATM noninvasive tail-3 cuff system. Treatments resulted in a mean plasma concentration (and 95% confidence interval) of 32.29 (16.71 to 47.74) and 25.13 ng/ml (7.81 to 42.46) for carvedilol and R-carvedilol, respectively. Only racemic carvedilol statistically reduced mean systolic blood pressure from the baseline (P=0.0367) while R-carvedilol did not (FIG. 1). This data are consistent with published study of oral R-carvedilol in mice at lower dose (1.6 mg/kg). The invention being thus described, it will be evident that the same may be varied in many ways by a routineer in the applicable arts. Such variations are not to be regarded as a departure from the spirit and scope of the invention and all such modifications are intended to be included within the scope of the claims.

### REFERENCES

Lin, C. S., Lin, W. S., Lin, C. L., and Kao, C. H. (2015). Carvedilol use is associated with reduced cancer risk: A nationwide population-based cohort study. Int J Cardiol 184, 9-13.
Chang, A., Yeung, S., Thakkar, A., Huang, K. M., Liu, M. M., Kanassatega, R. S., Parsa, C., Orlando, R., Jackson, E. K., Andresen, B. T., and Huang, Y. (2015). Prevention of skin carcinogenesis by the beta-blocker carvedilol. Cancer Prev Res (Phila) 8, 27-36.
Huang, K. M., Liang, S., Yeung, S., Oiyemhonlan, E., Cleveland, K. H., Parsa, C., Orlando, R., Meyskens, F. L., Jr., Andresen, B. T., and Huang, Y. (2017). Topically Applied Carvedilol Attenuates Solar Ultraviolet Radiation Induced Skin Carcinogenesis. Cancer Prev Res (Phila) 10, 598-606.
Cleveland, K. H., Liang, S., Chang, A., Huang, K. M., Chen, S., Guo, L., Huang, Y., and Andresen, B. T. (2019). Carvedilol inhibits EGF-mediated JB6 P+colony formation through a mechanism independent of adrenoceptors. PLOS One 14, e0217038.
Cleveland, K. H., Yeung, S., Huang, K. M., Liang, S., Andresen, B. T., and Huang, Y. (2018). Phosphoproteome profiling provides insight into the mechanism of action for carvedilol-mediated cancer prevention. Mol Carcinog 57, 997-1007.
Liang, S., Shamim, M. A., Shahid, A., Chen, M., Cleveland, K. H., Parsa, C., Orlando, R., Andresen, B., and Huang, Y. (2021). Prevention of skin carcinogenesis by the non-beta-blocking R-carvedilol enantiomer. Cancer Prev Res (Phila).
Stoschitzky, K., Koshucharova, G., Lercher, P., Maier, R., Sakotnik, A., Klein, W., Liebmann, P. M., and Lindner, W. (2001). Stereoselective effects of (R)- and(S)-carvedilol in humans. Chirality 13, 342-346.
Zhang, J., Zhou, Q., Smith, C. D., Chen, H., Tan, Z., Chen, B., Nani, A., Wu, G., Song, L. S., Fill, M., Back, T. G., and Chen, S. R. (2015). Non-beta-blocking R-carvedilol enantiomer suppresses Ca2+waves and stress-induced ventricular tachyarrhythmia without lowering heart rate or blood pressure. Biochem J 470, 233-242.

What is claimed is:

1. A drug for prophylactic use in humans having an increased risk of cancer, comprising:
carvedilol or R-carvedilol. in a clinically effective amount, being administered to a human being at increased risk of cancer;
whereby risk of cancer is reduced.

2. The drug of claim 1, wherein said carvedilol prevents ultraviolet (UV) radiation induced skin cancer by attenuating DNA damage, reducing inflammation and reversing immunosuppression

3. The drug of claim 1, wherein the healthy individuals with increased risks of cancer include any of: individuals with white skin tone, heavy smokers, and individuals with a weakened immune system.

4. The drug of claim 1, wherein said carvedilol prevents chemical carcinogen-induced lung cancer development.

5. A method for reducing risk of cancer in humans having an increased risk of cancer, comprising the steps of:
providing carvedilol or R-carvedilol;
administering said carvedilol or said R-carvedilol. in a clinically effective amount, to a human being at increased risk of cancer;
whereby risk of cancer is reduced.

6. The method of claim 5, comprising the step of using said carvedilol or said R-carvedilol to prevent ultraviolet (UV) radiation induced skin cancer by attenuating DNA damage, reducing inflammation and reversing immunosuppression.

7. The method of claim 5, comprising the step of providing said carvedilol or said R-carvedilol to healthy individuals with increased risks of cancer which include any of: individuals with white skin tone, heavy smokers, and individuals with a weakened immune system.

8. The method of claim 5, wherein said carvedilol prevents chemical carcinogen-induced lung cancer development.
